(19) United States
(12) Patent Application Publication
(10) Pub. No.: US 2022/0055360 A1
(43) Pub. Date: Feb. 24, 2022

(54) TATTOOABLE ELEMENT FOR DECORATING FASHION OR DECORATIVE OBJECTS AND HUMAN LIMBS

(71) Applicants: Eddi Drammeh Mora, Sabadell (Barcelona) (ES); Jordi Mora Saez, Benidorm (Alicante) (ES)

(72) Inventors: Eddi Drammeh Mora, Sabadell (Barcelona) (ES); Jordi Mora Saez, Benidorm (Alicante) (ES)

(21) Appl. No.: 17/312,171

(22) PCT Filed: Dec. 10, 2019
(86) PCT No.: PCT/ES2019/070838

§ 371 Date: Nov. 8, 2021

(30) Foreign Application Priority Data
Dec. 10, 2018 (ES) U201831895

(51) Int. Cl. B32B 27/28; B32B 27/12; B32B 7/12; B32B 1/08

(52) U.S. Cl. CPC B32B 27/283; B32B 27/12; A41D 27/10; B32B 1/08; B32B 7/12

(57) ABSTRACT

A tattooable element for decorating fashion or decorative objects and the limbs of the human body is provided that includes a polymer layer (2) suitable for tattooing and a reinforcing element (3) of the tattooable layer. The reinforcing element (3) can be an element that is separate from the polymer layer (2) and fixed to the polymer layer (2), or it can be integrated into the polymer layer (2). Optionally, the reinforcing element (3) prevents the needle from penetrating the tattooable element during the tattooing of the polymer layer (2). If used to decorate human limbs, it takes the form of an adaptable elastic tubular sheath.

### OBJECT OF THE INVENTION

The invention, as stated in the title of the present specification, relates to a tattooable element for decorating fashion or decorative objects that contributes advantages and features to the function intended, which are described in detail further on.

The object of the present invention relates, specifically, to a sheet element which, intended for being incorporated in fashion or decorative objects, such as footwear, items of clothing, fashion accessories, such as belts, bags, wallets, canvasses, tapestries, etc., and formed from a polymer layer attached to a reinforcing element such as a cloth or fibreglass base, where this latter part may or may not be part of the object itself, provides an ideal surface for being able to be tattooed, with any of the techniques conventionally used for directly tattooing skin, with optimal results in terms of execution and outcome, allowing for customised decoration, with any chosen image, of the object in which the element is implemented, either factory-made in that manner or as an added feature.

### FIELD OF APPLICATION OF THE INVENTION

The field of application of the present invention falls within the sector of the industry dedicated to the manufacture of fashion garments and objects, such as footwear, items of clothing, bags, etc., particularly focusing on the field of decorative elements for said type of objects.

### BACKGROUND OF THE INVENTION

As is known, tattoos are drawings etched onto the skin by means of a technique that involves the use of special needles to introduce colouring substances beneath the epidermis, providing a very specific and particular aesthetic result.

Synthetic skin or synthetic reproductions of parts of the body made from silicone for the practice of tattooing are also known. However, synthetic skin easily tears and as a consequence cannot be stitched.

The objective of the present invention is, therefore, to provide a means for allowing the incorporation of said type of images in fashion objects, such as footwear, items of clothing or others, or in decorative objects such as canvasses, tapestries, cushions, etc., so as to allow users to customise them with tattoos chosen by said users.

However, applying this same technique on surfaces other than the skin requires the development of a special surface which allows the tattoo to be executed in a suitable manner, for which purpose the element of the present invention has been developed.

### DESCRIPTION OF THE INVENTION

The tattooable element for decorating fashion or decorative objects proposed by the invention is configured as an optimal solution, since, based on its implementation, the aforementioned objectives are satisfactorily and unquestionably achieved, with the characterising details making it possible and suitably distinguishing it being included in the final claims attached to the present description.

More specifically, what the invention proposes, as indicated above, is a sheet element designed as an element to decorate, presenting the particularity that it is tattooable, in addition to being susceptible to being handled, sewn and stitched, where it can be implemented in the industrial production system for any type of footwear (shoes, trainers, boots, etc.) and/or any type of item of clothing (jackets, pullovers, blazers, T-shirts, pants, etc.) or other fashion accessories (belts, bags, wallets, etc.) or decorative objects (canvasses, chair or sofa tapestries, cushions, etc.), or as an accessory that can be sold as an element that is separate and can be fixed to any of said objects once they have been manufactured and already existing on the market.

To that end and more specifically, the decorative tattooable element is essentially configured from a polymer layer suitable for being tattooed and a reinforcing element of the polymer layer.

This reinforcing element improves the features of the polymer layer allows the handling, sewing and stitching of the tattooable element, allowing same to be incorporated in the fashion object or article of clothing in question, even constituting the actual fabric for producing the article of clothing or being a part thereof.

Additionally and in the event of the polymer layer tearing, the reinforcing element allows the pieces of the polymer layer to remain in their original position, thus preventing the figure of the tattoo from losing its original shape.

In a preferred embodiment, the reinforcing element is an element that is separate from the polymer layer and fixed to the polymer layer.

The reinforcing element can be a fabric or fibreglass. Both the fabric and the fibre limit the elastic properties of polymers such as silicone, thus allowing them to be susceptible to stitching and implemented in fashion and/or decorative objects.

The reinforcing element can be fixed to the polymer layer by means of glue or fusing by applying heat and pressure on the reinforcing element against the polymer layer.

Alternatively, in another preferred embodiment the reinforcing element is integrated into the polymer layer.

Preferably, the reinforcing element is a mesh integrated into the polymer layer or an array of fibres integrated into the polymer layer. The advantage is that when the polymer layer is fused with the fabric or fibre mesh, both fused elements work like a single piece, adding resistance to the stitching and increasing the durability in other fields of application.

Regardless of how the reinforcing element is carried out, the polymer layer is preferably made of silicone. Silicone offers a smooth surface suitable for being tattooed with optimal results in terms of definition, durability and artistic execution.

Regardless of how the reinforcing element is carried out, the polymer layer preferably has a thickness of more than 1 mm, and even more preferably a thickness of more than 3 mm.

In any case, the main advantage of the tattooable element for decorating fashion or decorative objects provides, from a commercial approach, a technology that is very attractive in terms of value, according to current trends, for customising and adding a distinctive quality to footwear, articles of clothing or other fashion accessories.

### DESCRIPTION OF THE DRAWINGS

To complete the description provided herein, and for the purpose of helping to make the features of the invention more readily understandable, this description is accompanied by drawings constituting an integral part of the same, which by way of illustration and not limitation represents the following:

FIG. 1 shows a schematic perspective view of the two sheet layers forming the tattooable element for decorating fashion or decorative objects, object of the invention, represented in an exploded view;

FIG. 2 shows a schematic section view of the tattooable element according to the invention, in this case represented once the two sheet layers it comprises have been attached to one another;

FIG. 3 shows a schematic perspective view of the tubular-shaped tattooable element;

FIG. 4 shows a schematic perspective view of the tubular-shaped tattooable element fixed on a user's forearm;

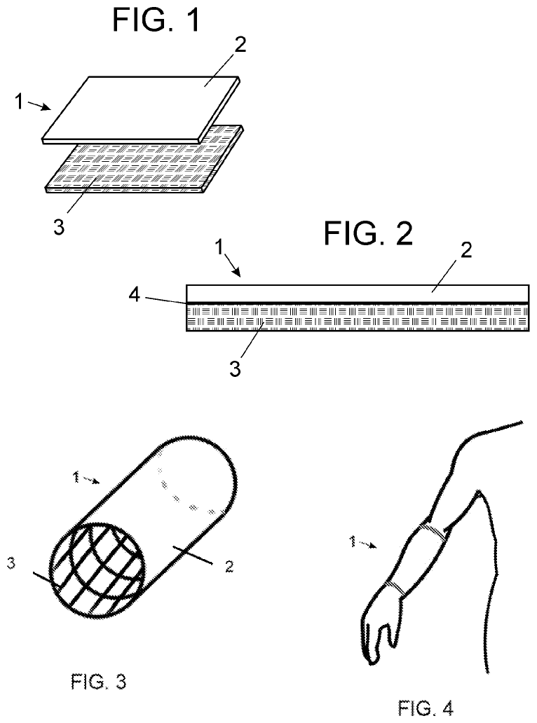

### PREFERRED EMBODIMENT OF THE INVENTION

In view of the aforementioned figures, and in accordance with the adopted numbering, one may observe therein a non-limiting exemplary embodiment of the proposed tattooable element for decorating fashion or decorative objects, comprising what is described in detail below.

Thus, as can be observed in said figures, the element (1) in question is essentially configured from a sheet element formed by two layers of different material: an upper silicone layer (2), providing the smooth surface suitable for tattooing with any conventional technique, and preferably having a thickness of about 1 mm, and a lower layer (3) acting as a base for the upper layer (2), where said lower layer (3) may be made of either fabric or fibreglass.

Furthermore, the upper silicone layer (2) is preferably made of medical grade silicone (RTV (Room Temperature Vulcanisation)), since in addition to being hypoallergenic, it does not contain latex or toxins, or any type of additive, nor is it porous, whereby its use in articles of clothing in contact with the skin is feasible, and especially, it provides an ideal surface for tattooing, with optimal results of definition and resistance.

It should be pointed out that the lower base layer (3) is preferably attached to the upper silicone layer (2) by means of any fixing system, for example a glue layer (4), which may be fused with same, for example, by means of applying heat.

Furthermore, the element (1) may be trimmed in order to give it the desired shape, and coupled to the object by means of stitching, gluing, or any other fixing means, even by means of a removable system in order to take it off and put it on at will.

In a preferred embodiment, the tattooable element is tubular-shaped. In an even more preferred embodiment, the tattooable element is elastic. This embodiment allows the tattooable element to be fixed on a user's body, for example on the forearm as shown in FIG. 4, allowing the tattoo artist to have a tattooing experience very similar to the real experience.

In a preferred embodiment, the reinforcing element (3) prevents the needle from penetrating the tattooable element during the process of tattooing the polymer layer (2). The user can thereby be tattooed without any apprehension about the needle penetrating the tattooable element and being able to damage human skin.

Having sufficiently described the nature of the present invention, as well as the ways of implementing it, it is not considered necessary to extend its explanation for any person skilled in the state of the art to understand its scope and the advantages which derive from it, specifying that, within its essential nature, it can be carried out in other embodiments that differ in detail from the one provided by way of example, and which are also covered by the requested protection, provided that they do not alter, change or modify its fundamental principle.

1. The tattooable element for decorating fashion or decorative objects, characterised by comprising a polymer layer (2) suitable for tattooing and a reinforcing element (3) of the tattooable layer.

2. The tattooable element according to claim 1, characterised in that the reinforcing element (3) is an element that is separate from the polymer layer (2) and fixed to the polymer layer (2).

3. The tattooable element according to claim 2, characterised in that the reinforcing element (3) is made of fabric.

4. The tattooable element according to claim 2, characterised in that the reinforcing element (3) is made of fibreglass.

5. The tattooable element according to claim 1, characterised in that the reinforcing element (3) is fixed to the polymer layer (2) by means of glue (4).

6. The tattooable element according to claim 1, characterised in that the reinforcing element (3) is fixed to the polymer layer (2) by means of fusing.

7. The tattooable element according to claim 1, characterised in that reinforcing element (3) is integrated within the polymer layer (2).

8. The tattooable element according to claim 7, characterised in that the reinforcing element (3) is a mesh.

9. The tattooable element according to claim 7, characterised in that the reinforcing element (3) is fibres.

10. The tattooable element according to claim 1, characterised in that the polymer layer (2) is made of silicone.

11. The tattooable element according to claim 1, characterised in that the polymer layer (2) has a thickness of more than 1 mm.

12. The tattooable element according to claim 10, characterised in that the polymer layer (2) has a thickness of more than 3 mm.

13. The tattooable element according to claim 1, characterised in that the reinforcing element (3) prevents the needle from penetrating the tattooable element during the process of tattooing the polymer layer (2).

14. The tattooable element according to claim 1, characterised in that it is tubular-shaped.

15. The tattooable element according to claim 1, characterised in that it is elastic and allows being adapted to the support on which the tattooable element is fixed.
